(19) United States
(12) Patent Application Publication
(10) Pub. No.: US 2023/0270407 A1
(43) Pub. Date: Aug. 31, 2023

(54) MEDICAL DEVICES, SYSTEMS, AND METHODS INCORPORATING THE SAME

(71) Applicant: XENTER, INC., Draper, UT (US)

(72) Inventors: Richard J. LINDER, Sandy, UT (US); Nathan J. KNIGHTON, Centerville, UT (US); Scott HEULER, LakeWood Ranch, FL (US); Fernando A. SOSA, Vista, CA (US)

(21) Appl. No.: 18/114,141

(22) Filed: Feb. 24, 2023

Related U.S. Application Data
(60) Provisional application No. 63/314,392, filed on Feb. 26, 2022.

(51) Int. Cl. A61B 8/00; A61B 8/12

(52) U.S. Cl. CPC A61B 8/4416; A61B 8/463; A61B 8/12; A61M 2025/0002

(57) ABSTRACT

Medical devices, medical systems, and related methods are provided which may be used in detecting and treating anomalies in a vessel (e.g., in an artery). In one embodiment, a guidewire is provide with multiple sensors spaced apart from one another in a desired spacing. The sensors may each be configured to simultaneously detect a pressure within the vessel at their individual location within a single, common heartbeat of a patient. In one embodiment, information from the sensors may be mapped with other data or information (e.g., imaging data) to assist a healthcare professional in determining if interventional treatment is desired and, if so, what treatment may be most effective.

### CROSS-REFERENCE TO RELATED APPLICATIONS

This application claims the benefit of U.S. Provisional Patent Application No. 63/314,392, entitled MEDICAL DEVICES, SYSTEMS AND METHODS INCORPORATING SAME, filed on Feb. 26, 2022, the disclosure of which is hereby incorporated in its entirety by reference herein.

### BACKGROUND

The present disclosure relates generally to medical devices incorporating sensors, as well as systems and methods which may incorporate such devices. Additionally, the present disclosure relates to devises and systems for providing information to a healthcare practitioner who may be utilizing such devices, systems or practicing associated methods.

In one, non-limiting example, such medical devices may include intraluminal devices, such as guidewires and catheters, having various sensors for simultaneous and/or continuous measuring of one or more physiological parameters and imaging.

Guidewire devices are often used to lead or to guide catheters or other interventional devices to a targeted anatomical location within a patient's body. For example, guidewires may be passed into and through a patient's vasculature in order to reach the target location, which may be, for example, at or near the patient's heart or brain. Radiographic imaging is conventionally utilized to assist in navigating a guidewire to the targeted location. Guidewires are available with various outer diameter sizes. Widely utilized sizes include 0.010, 0.014, 0.016, 0.018, 0.024, and 0.035 inches in diameter, for example, though they may also be smaller or larger in diameter.

In some instances, a guidewire may be used to gather physiological information from within a patient. For example, so-called “pressure wires” conventionally incorporate a single pressure sensor to detect the blood pressure within a blood vessel of a patient.

In many instances, a guidewire is placed within the body during the interventional procedure so that it can be used to guide one or more catheters or other interventional devices to the targeted anatomical location. For example, a catheter can be guided to a targeted location and, once in place, be used to image the targeting location, to aspirate clots or other occlusions, or to deliver drugs, stents, embolic devices, radiopaque dyes, or other devices or substances for treating the patient.

These types of interventional devices can include sensors located at the distal end in order to provide added functionality to the device. For example, intravascular ultrasound (IVUS) is an imaging technique that utilizes a catheter with an ultrasound imaging sensor attached to the distal end. Ultrasound may be utilized to image within targeted vasculature (typically the coronary arteries).

There are several challenges associated with using sensors with intraluminal devices. For example, such interventional devices have very limited space to work in, given the stringent dimensional constraints involved. Moreover, integrating the sensors with the interventional device in a way that maintains effective functionality can be challenging.

The use of such interventional devices can also be challenging due to the need to manage several long lengths of wires and other components, including guidewires, power cables, data wires, and the like. Care must be taken with respect to what is allowed in the sterile field and when certain devices or components can be removed. Additional staff is often required simply to manage such wires, cables, and components.

As such, there is an ongoing need for improved medical devices that effectively integrate sensors and can help provide data in a more efficient manner and/or provide data previously unobtainable in a practical manner.

### SUMMARY

The present disclosure provides medical devices, systems using medical devices, and methods using such devices and/or systems. In some embodiments, a medical device may include a guidewire having one or more sensors or other electronic components. In some embodiments, a system may include a guidewire used in conjunction with a display system incorporating a user interface.

In one embodiment, a medical device comprises an elongated member having a distal portion configured to be disposed in an artery. A first group of sensors, comprising at least two sensors, is associated with the distal portion of the elongated member. The first group of sensors is configured to detect a physiological parameter at at least two different locations within the artery during a single heartbeat (e.g., from the beginning of ventricular diastole through the end of ventricular systole and/or from one P-wave to the beginning of the next) of a patient.

In one embodiment, the elongated member comprises a guidewire.

In one embodiment, the at least two sensors are spaced apart between approximately 0.5 cm and approximately 2.0 cm using a center-to-center spacing.

In one embodiment, the group of sensors includes eleven sensors spaced approximately 1.0 cm apart from each other using a center-to-center spacing.

In one embodiment, the group of sensors includes a plurality of pressure sensors.

In one embodiment, the device further comprises a proximal pressure sensor associated with the guidewire and positioned proximally of the first group of sensors.

In one embodiment, the proximal pressure sensor is configured to detect a pressure within the artery during the same single heartbeat as the first group of sensors.

In another embodiment of the present disclosure, a system is provided that comprises a guidewire having a group of pressure sensors associated with a distal portion of the guidewire and a catheter having at least one ultrasound transducer configured to provide an ultrasound image of an artery. The system further includes a display having: an angiogram image of the artery, a pressure map of pressure measurements received from the group of pressure sensors, and a longitudinal ultrasound image of the artery aligned with the pressure map.

In one embodiment, the display further comprises a first cross-sectional ultrasound image of the artery.

In one embodiment, the display further comprises a second cross-sectional ultrasound image of the artery.

In one embodiment, the first cross-sectional ultrasound image is of an area of the artery including a lesion.

In one embodiment, the second cross-sectional ultrasound image is a reference image for comparison with the first cross-sectional image.

In one embodiment, the system further comprises annotations overlaid on the angiogram image, the annotations including indications of suggested landing zones for a stent.

In one embodiment, the system further includes indicia representing a suggested stent length positioned in alignment with an indication of a lesion on the longitudinal ultrasound image.

In one embodiment, the group of pressure sensors includes at least two sensors spaced apart between approximately 0.5 cm and approximately 2.0 cm using a center-to-center spacing.

In one embodiment, the group of pressure sensors includes eleven sensors spaced approximately 1.0 cm apart from each other using a center-to-center spacing.

In one embodiment, the group of pressure sensors is configured to detect a physiological parameter at at least two different locations within the artery during a single heartbeat of a patient.

In one embodiment, the system further comprises a proximal pressure sensor.

In one embodiment, the proximal pressure sensor is configured to detect a pressure within the artery during the same single heartbeat as the first group of sensors.

In one embodiment, the proximal pressure sensor is associated with the guidewire and located proximally of the group of pressure sensors.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The foregoing and other advantages of the invention will become apparent upon reading the following detailed description and upon reference to the drawings in which:

FIG. 1A illustrates a guidewire system according to an embodiment of the present disclosure;

FIG. 1B is a view of a distal portion of a guidewire according to an embodiment of the present disclosure;

FIG. 2 illustrates a catheter system according to an embodiment of the present disclosure;

FIG. 3 depicts a display screen containing information and tools available to healthcare providers performing or otherwise assisting with a medical procedure.

### DETAILED DESCRIPTION

Various embodiments described herein are directed toward the incorporation of electronic devices (e.g., sensors and transducers) into medical devices, systems incorporating such medical devices, and related methods.

In some embodiments, devices associated with cardiovascular, neurovascular, and endovascular procedures are provided having sensors integrated therewith. For example, guidewires or catheters may include sensors, transducers, or other electronic or optical components integrated into the structure for detecting, imaging or measuring physiological data (e.g., pressure, flow rate, etc.), providing imaging data (e.g., ultrasound images), and providing that data to a healthcare provider in real time during an associated procedure.

In some embodiments, other sensors or electronic elements are associated with the device. For example, sensors configured to detect the presence of biological components may be incorporated into or otherwise associated with the device. In some embodiments, a transmission element (e.g., an antenna structure) may be integrated with the device for providing wireless transmission of data.

Some non-limiting examples of medical devices that may incorporate such sensors (as well as discussions of associated systems and methods) include those described in: U.S. patent application Ser. No. 17/205,964, entitled “Guidewire for Imaging and Measurement of Pressure and Other Physiological Parameters” and filed on Mar. 18, 2021; U.S. patent application Ser. No. 17/205,854, entitled “Catheter for Imaging and Measurement of Pressure and Other Physiological Parameters” and filed on Mar. 19, 2021; U.S. patent application Ser. No. 17/205,754, entitled “Operatively Coupled Data and Power Transfer Device for Medical Guidewires and Catheters with Sensor” and filed on Mar. 18, 2021; U.S. patent application Ser. No. 17/205,614, entitled “Signal Conducting Device for Concurrent Power and Data Transfer to and From Un-wired Sensors Attached to a Medical Device” and filed on Mar. 18, 2021; U.S. patent application Ser. No. 17/979,629, entitled “Data and Power Transfer Devices for Use with Medical Devices and Related Methods” and filed Nov. 2, 2022; U.S. Provisional Patent Application No. 63/394,591, entitled “Medical Devices, Systems, and Methods Including Power and Data Transfer” and filed on Aug. 2, 2022; and U.S. Provisional Patent Application No. 63/442,982 entitled “Medical Devices, Medical Systems, and Related Methods” and filed Feb. 2, 2023, the disclosures of which are each incorporated by reference herein in their entireties.

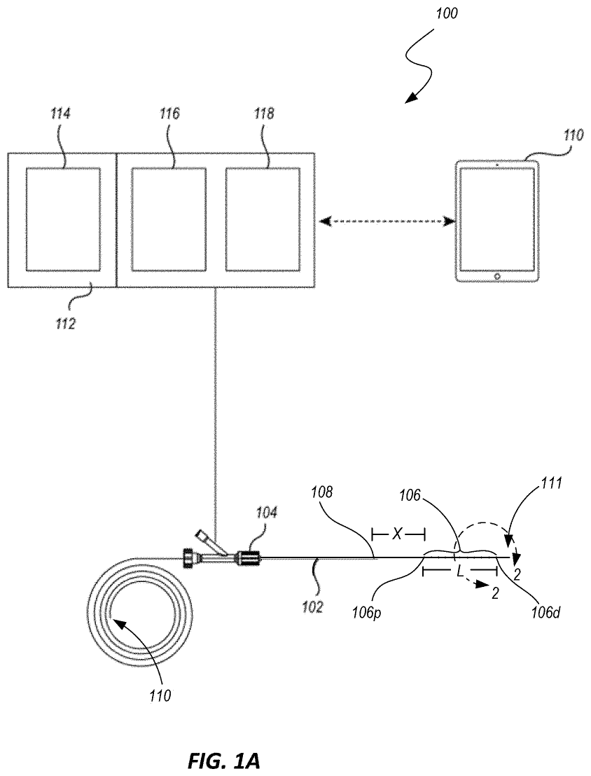

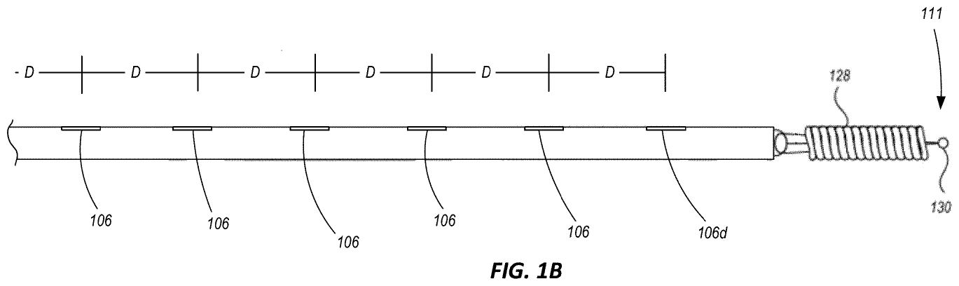

Referring to FIG. 1A, a guidewire system 100 is illustrated according to an embodiment of the present disclosure. As shown, the guidewire system 100 includes a wire 102, a proximal device 104 which, in some embodiments, may include a hemostatic valve. The system 100 further includes a plurality of longitudinally spaced sensors 106 associated with a distal end of the wire 102, one or more additional sensors 108 proximally located from the distal group of longitudinally spaced sensors 106, and a control unit 112 (shown enlarged and in schematic form) that includes a power source 114, data signal processor 116, and optionally a transmitter 118. The system 100 may further include an external device 110 (e.g., a stationary or handheld computer, a stationary or handheld display, a tablet computer, a smart phone, or other input and/or output device) which may be in wired or wireless communication with the transmitter 118. In some embodiments, the control unit 112 may be disposed, entirely or partially, within a body or housing of the proximal device 104. In other embodiments, the control unit may be separate from proximal device but configured for operational communication with the wire and its various components. In other embodiments, the control unit 112 may be physically separate from, but electrically coupled with components in or otherwise associated with the proximal device 104.

In some embodiments, the proximal device 104 may be configured as a data and/or power transfer device. For example, the proximal device 104 may be configured for electrical coupling with the wire 102 to provide power to sensors or other electronics associated with the wire and to receive various signals provided by the sensors or other electronic components. Such electrical coupling between the proximal device 104 and the wire 102 may be accomplished via direct conductive connection or via an electronic field (e.g., through a capacitive coupling).

The data signal processor 116 may be configured to receive sensor data signals, sent through the wire 102, from one or more sensors 106 (which may be located and oriented in specific patterns as individual sensors, or may be configured as identifiable sensor arrays) associated with the wire 102. The power source 114 may be configured to transmit power through the wire 102 to power the one or more sensors 106 and/or other components of the wire 102. The power source 114 may include an on-board power source, such as a battery or battery pack, and/or may include a wired connection to an outside power source. The one or more sensors 106 may be located at any suitable position on the wire 102 but are disposed at the distal section, which is expected to reach the targeted anatomy, in the depicted embodiment. As used herein, the “distal section” or “distal portion” refers to the distal-most 30 cm of the device, the distal-most 20 cm of the device, the distal-most 15 cm of the device, the distal-most 10 cm of the device, or to a range using any two of the foregoing values as endpoints. In some embodiments, the “intermediate section” may be considered as roughly the middle third of the device, and the “proximal section” or “proximal portion” may be considered as roughly the proximal third of the device.

In one embodiment, the guidewire system 100 is configured to send power to the sensors 106 through individual traces or other electrical conductors (e.g., routed along an outer surface and/or attached to an outer surface of the wire 102), and data signals from the sensors may be transmitted via separate transmission structures or schemes. For example, data may be transmitted by additional traces or electrical conductors (e.g., routed along an outer surface and/or attached to an outer surface of the wire 102), by optical transmission, and/or by wireless transmission.

In another embodiment, the guidewire system 100 is configured to send power and data signals through the actual wire 102 itself (e.g., the structural core wire) rather than through traces or through separate, discrete electrical conductors. In some embodiments, multiple power and/or data signals (e.g., data signals from multiple sensors 106) can be sent through the wire 102 simultaneously. Power and/or data signals can also be sent in a “continuous” fashion. That is, the power and/or data signals can have a sufficiently high sampling rate such that the information is provided to the user within time frames that are practically “real-time.” Nonlimiting examples of managing power and/or data signals, along with associated components and systems, as set forth in the previously incorporated U.S. patent applications.

The wire 102 of the guidewire system 100 is configured for insertion into the body of a subject. The subject is typically a human, but in other implementations may be a non-human mammal or even non-mammalian animal. Any suitable route of administration may be utilized, depending on particular preferences and/or application needs. Common routes include femoral, radial, and jugular, but the guidewire system 100 may utilize other access routes as needed.

The wire 102 has a proximal end 110 and a distal end 111. The length of the wire 102 may vary according to particular application needs and targeted anatomical area. As an example, the wire 102 may have an overall length from proximal end 110 to distal end 111 of about 50 cm to about 350 cm, more commonly about 200 cm, depending on particular application needs and/or particular anatomical targets. The wire 102 may have a size such that the outer diameter (e.g., after application of other outer members) is about 0.008 inches to about 0.040 inches, though larger or smaller sizes may also be utilized depending on particular application needs. For example, particular embodiments may have outer diameter sizes corresponding to standard guidewire sizes such as 0.010 inches, 0.014 inches, 0.016 inches, 0.018 inches, 0.024 inches, 0.035 inches, 0.038 inches, or other such sizes common to guidewire devices. The wire 102 may be formed from materials comprising stainless steel or other metal or alloy having similar appropriate properties. In some embodiments, the wire 102 may be formed of or may comprise a conductive material of appropriate mechanical properties.

Referring to FIG. 1B, one or more sensors 106 of the guidewire system 100 may include, for example, a pressure sensor, a flow sensor, an imaging sensor, a component detection sensor, or combinations thereof. Additionally, while generally referred to as a “sensor” in discussing various embodiments throughout, such “sensor” components (e.g., sensor 106 and 108) may comprise or otherwise be associated with transducers or other electrical, electromechanical, electrochemical, or optical components and may be configured as input devices, output devices, or both. In other words, sensor components described herein may include or at least be a part of transducers that are capable of transmitting and/or receiving a specified type of signal. For example, such sensors may include ultrasound transducers, optical emitters (e.g., light emitting diodes (LEDs)), optical sensors (e.g., photo diodes) and the like.

The one or more sensors may additionally, or alternatively, be configured to sense the presence of biological components or measure physiological parameters in the targeted anatomical location (e.g., in the blood). Example biological components that may be detected/measured include sugar levels, pH levels, CO2 levels (CO2 partial pressure, bicarbonate levels), oxygen levels (oxygen partial pressure, oxygen saturation), temperature, and other such substrates and physiological parameters. The one or more sensors may be configured to sense the presence, absence, or levels of biological components such as, for example, immune system-related molecules (e.g., macrophages, lymphocytes, T cells, natural killer cells, monocytes, other white blood cells, etc.), inflammatory markers (e.g., C-reactive protein, procalcitonin, amyloid A, cytokines, alpha-1-acid glycoprotein, ceruloplasmin, hepcidin, haptoglobin, etc.), platelets, hemoglobin, ammonia, creatinine, bilirubin, homocysteine, albumin, lactate, pyruvate, ketone bodies, ion and/or nutrient levels (e.g., glucose, urea, chloride, sodium, potassium, calcium, iron/ferritin, copper, zinc, magnesium, vitamins, etc.), hormones (e.g., estradiol, follicle-stimulating hormone, aldosterone, progesterone, luteinizing hormone, testosterone, thyroxine, thyrotropin, parathyroid hormone, insulin, glucagon, cortisol, prolactin, etc.), enzymes (e.g., amylase, lactate dehydrogenase, lipase, creatine kinase), lipids (e.g., triglycerides, HDL cholesterol, LDL cholesterol), tumor markers (e.g., alpha fetoprotein, beta human chorionic gonadotrophin, carcinoembryonic antigen, prostate specific antigen, calcitonin), and/or toxins (e.g., lead, ethanol).

In the embodiment shown in FIGS. 1A and 1B (which are not to be considered to-scale representations), multiple sensors 106 are arranged longitudinally along a length of a distal portion of the wire 102. In one embodiment, the sensors 106 are configured as pressure sensors (e.g., a capacitive type sensor or a piezo type sensor). Each of the longitudinally spaced, distal sensors 106 (referred to herein as the “distal group of sensors 106” for convenience) may be spaced from one another a desired distance D and may cover (or extend through) a specified length L along the longitudinal length of the wire 102. For example, in one embodiment, there may be eleven sensors 106, each spaced a center-to-center distance D of approximately 1 centimeter (cm) apart from adjacent sensors and covering a total length L of approximately 10 cm between the distal-most sensor 106d and the proximal-most sensor 106p of the distal group of sensors. In other embodiments, the values of D and L may be different. For example, D may be, without limitation, 0.5 cm, 1.0 cm, 1.5 cm, 2.0 cm, or 2.5 cm (or larger or smaller), or it may be a distance in any range that includes such example distances as endpoints (e.g., 0.5 cm to 1.0 cm; 1.0 cm to 2.0 cm; or 0.5 cm to 2.5 cm). In another example, L may be, without limitation, 2.5 cm, 5 cm, 7.5 cm, 10 cm, 12.5 cm, 15 cm, 17.5 cm, or 20 cm (or larger or smaller), or it may be any range including such example lengths as endpoints (e.g., 2.5 cm to 15 cm; 10 cm to 20 cm; or 5 cm to 20 cm). Additionally, the number of sensors may be different from that depicted in FIGS. 1A and 1B, and may include, without limitation, two sensors in the distal group of sensors 106, five or more sensors, ten or more sensors, fifteen or more sensors, twenty or more sensors, twenty-five or more sensors, or thirty or more sensors in various embodiments, or a range that includes such example quantities as endpoints.

It is noted that while the embodiment shown in FIGS. 1A and 1B depict sensors 106 that are evenly spaced (i.e., with D being the same between adjacent sensors), other embodiments may include sensors having a varied distance between them (i.e., at least one D may be different from another D). For example, the centermost sensors may be closer together than are adjacent sensors near the ends of the sensor set, or vice versa. The distance between sensors may be determined, in part by the resolution of measurements desired as well as the type of sensor being employed (e.g., pressure sensors, flow sensors, etc.).

The proximal sensor 108 may be positioned a distance X from the proximal-most sensor 106p of the group of longitudinally spaced sensors 106. In one embodiment, the distance X may be substantially equal to the distance D. In other embodiments, the distance X may be some multiplier of the distance D. For example, considering D to be approximately 1 cm, the distance X may be 2 cm, 5 cm, 10 cm, 15 cm, or 20 cm, or within a range using any of these example distances (including any distance D disclosed herein) as an endpoint.

Still referring to FIGS. 1A and 1B, a coil 128 and/or atraumatic tip 130 may be located at the distal end 111. The coil 128 may be a single coil or multiple connected or interwoven coils. Additionally, or alternatively, a polymer material may be positioned on or applied to the distal section of the wire 102. The atraumatic tip 130 may include a sphere or other curved geometry to protect against potential trauma that might otherwise be caused by the distal end of the wire 102. The atraumatic tip 130 may be formed from a polymer adhesive material and/or solder, for example.

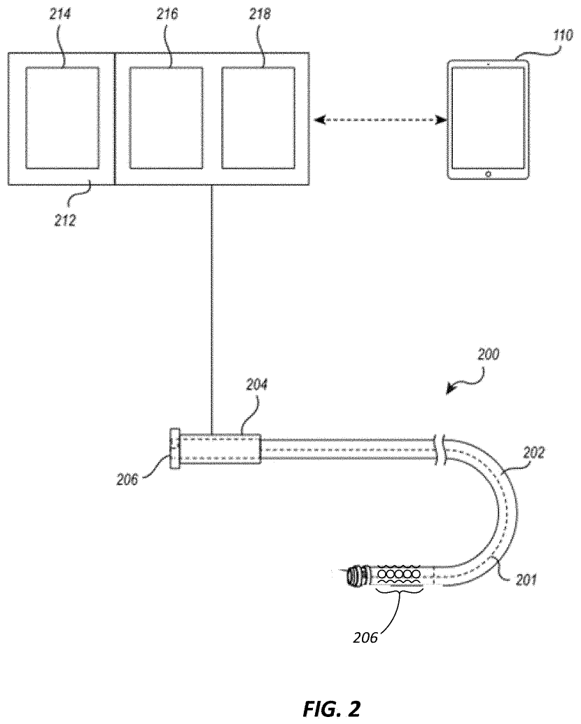

Referring now to FIG. 2, an overview of a catheter system 200 according to an embodiment of the present disclosure is provided. The catheter system 200 may be similar to the guidewire system 100 in many respects, and the above description related to the guidewire system 100 is also applicable here except where differences are specified. The catheter system 200 includes a catheter 202 and a proximal device 204 (which may also be referred to herein as “the power and data coupling device 204” or just “the coupling device 204”). The coupling device 204 includes a control unit 212 (shown enlarged and in schematic form) that includes a power source 214, a data signal processor 216, and optionally a transmitter 218. The transmitter 218 enables wireless communication to the external device 110 (or multiple of such devices).

The data signal processor 216 is configured to receive sensor data signals, sent through the catheter 202, from one or more sensors 206 associated with the catheter 202. The power source 214 is configured to transmit power through the catheter 202 (e.g., via the proximal device 204) to power the one or more sensors 206 and/or other components of the catheter 202. The power source 214 may include an on-board power source, such as a battery or battery pack, and/or may include a wired connection to an outside power source. The one or more sensors 206 may be located at any suitable position on the catheter 202 but will typically be disposed at the distal section of the catheter 202 expected to reach the targeted anatomy. Sensors 206 may be coupled to the catheter 202 by employing bonding, molding, co-extrusion, welding and/or gluing techniques, for example. Power wires and/or data lines 201 may extend along the length of the catheter 202 to the one or more sensors 206. As used herein, a “power line” and/or “data line” refer to any electrically conductive pathway (e.g., traces) within or on the medical device. Although multiple power and/or data lines 201 may be utilized, some embodiments may be configured to send both power and data on a single line and/or manage sensor data signals from multiple sensors on a single line. This reduces the number of lines that must be routed through the structure of the catheter 202 and more effectively utilizes the limited space of the device, as well as reducing the complexity of the device and the associated risk of device failure.

The proximal device 204 may include one or more ports to facilitate the introduction of fluids (e.g., medications, nutrients, nanoparticle colloidal solutions) into the catheter 202. The catheter 202 may be sized and configured to be temporarily inserted in the body, permanently implanted in the body, or configured to deliver an implant in the body. In one embodiment, the catheter 202 is a peripherally inserted central catheter (PICC) line, typically placed in the arm or leg of the body to access the vascular system of the body. The catheter 202 may also be a microcatheter, a central venous catheter, an IV catheter, coronary catheter, stent delivery catheter, balloon catheter, atherectomy type catheter, or IVUS (intravenous ultrasound) catheter or other imaging catheter. The catheter 202 may be a single or multi-lumen catheter.

One or more sensors 206 of the catheter system 200 may include a pressure sensor, a flow sensor, an imaging sensor (e.g., an ultrasound transducer), a component detection sensor, or combinations thereof, for example. Additionally, while generally referred to as a “sensor” in discussing various embodiments throughout, such “sensor” components (e.g., sensor 206) may comprise or otherwise be associated with transducers or other components and may be configured as input devices, output devices, or both.

The sensors 206 may be arranged in one or more a sensor arrays, wherein each sensor array includes a plurality of sensors 206 arranged in a desired pattern and are coordinated (e.g., through associated electronics or other computational components associated with the catheter system 200). In one embodiment, the sensors 206 include a plurality of ultrasound transducers (e.g., CMUT or PMUT transducers) configured to image the interior of a specified anatomy. For example, the catheter 202 may be configured as an IVUS catheter using ultrasound transducers to image a blood vessel or other anatomy.

In accordance with one embodiment of the present disclosure, the guidewire system 100 may be employed by inserting the guidewire 102 into an artery of a patient. The wire 102 may be positioned such that the distal-most sensor 106d is located distally of a lesion (or of multiple lesions) within an artery. Some of the other sensors may be located distally of the lesion, may be located coincident with or within the lesion, or may be located proximally of the lesion. In some embodiments, the wire 102 may be configured so that the proximal sensor 108 remains proximal of the lesion even though one or more sensors 106 of the distal sensor group are positioned distal of the lesion. Imaging (e.g., an angiogram) may be utilized in helping with the positioning of the guidewire 102 within the artery of the patient.

With the wire 102 positioned within the artery (or other anatomical structure), each of the sensors 106 and 108 may determine the blood pressure within the artery at their respective locations. In one embodiment, the proximal sensor 108 may be used to take a pressure reading within or near the aorta (or at least at a location that is proximal to the lesion being investigated) and may serve as a reference pressure (sometimes referred to as the aortic pressure). The other sensors 106 of the distal group of sensors may also provide blood pressure measurements at their specific locations. Each of the sensors 106 (and optionally 108 and/or other aortic pressure reading such as taken in a proximal device, as noted below) may take their pressure reading at their individual location simultaneously, or at least substantially simultaneously (e.g., all of the sensors 106 taking a respective measurement within less than one second, such as within a period of approximately 500 milliseconds, or within 100 milliseconds or even less). Thus, considering the pressure wave created by a heartbeat, a pressure reading (or even multiple pressure readings) may be taken by each sensor within the same cycle of a heartbeat to provide an accurate pressure map that extends longitudinally along an identified portion of the artery. Likewise, the proximal sensor 108 may take a pressure reading (or multiple pressure readings) during the same heart cycle as those of the distal sensor group. This may all be done while the guidewire 102 remains stationary within the artery (i.e., the guidewire does not need to be subjected to a “pullback” procedure). This provides an advantage over prior art methods of conducting a conventional pullback operation of the guidewire, wherein pressure readings are taken at different discrete times (usually over a period of several seconds), such that not all of the pressure measurements are taken within a common cycle of a single heartbeat.

In another embodiment, the proximal or so called “aortic pressure” may be additionally or alternatively measured at a location external to the body of a patient. For example, a sensor located in or otherwise associated with a proximal device (such as 104 or 204) may be used to provide a reference pressure for the distal group of sensors. A proximal device having a pressure sensor is described, for example, in U.S. patent application Ser. No. 17/979,629, entitled “Data and Power Transfer Devices for Use with Medical Devices and Related Methods” and filed Nov. 2, 2022, the disclosure of which is hereby incorporated in its entirety by reference herein.

As will be recognized by those of ordinary skill in the art, the pressure measurements taken with the distal group of sensors 106 may be compared to the reference measurement of the proximal sensor 108 (and/or an aortic pressure measured elsewhere) for purposes of assessing the health of the artery. For example, the measurement of each sensor 106 at a defined point in time may be compared to the measurement of the proximal sensor 108. Thus, a difference or a ratio of the measurements from each of the distal group of sensors 106, relative to the proximal sensor 108, may indicate the presence of and/or severity of a lesion. Various methods, such as fractional flow reserve (FFR) and/or any of a variety of non-hyperemic pressure ratios (NHPR—generally comprising a distal pressure (Pd) divided by the aortic pressure (Pa)) may be used.

In addition to obtaining pressure measurements using the guidewire system 100, the catheter system 200, when configured as an IVUS catheter, may be used to make an ultrasound image of the artery. In such cases, the catheter 202 may be “threaded” over the guidewire 102 to place an array of imaging sensors at the location of the lesion. In some embodiments, the catheter system 200 and the guidewire system 100 may include a common control unit (e.g., 112 or 212), or may share resources such as a common signal processing device (e.g., 116 or 216), a common power source (114 or 214) and/or a common display our output device (e.g., 110 or 210).

Additional sensors (not shown) associated with the catheter 202 and the guidewire 102 may be used to track the relative position of the catheter 202 and the guidewire 102 so that images taken with the catheter 202 may be correlated with pressure mappings from the guidewire 102. For example, a sensor in the catheter 202 may be used to detect individual “markers” or other sensors associated with the guidewire to determine their relative positions (e.g., similar to a linear encoder). In other embodiments, on or more sensors in the guidewire may be used to detect a marker of the catheter 202. In some embodiments, the sensors 106 and 108 may be used to detect the presence of the catheter, such as is described in the previously incorporated patent applications. In other embodiments, a common “proximal device” (e.g., 104 or 204) may be used with the guidewire system 100 and the catheter system 200, and the proximal device 104 or 204 may include sensors to track the relative positions of the catheter 202 and the guidewire 102. In yet other embodiments, markers located in the catheter 202 and/or guidewire 102 may be located using external detectors (e.g., a node positioned on a patient's skin at a strategic location) or by using external imaging techniques.

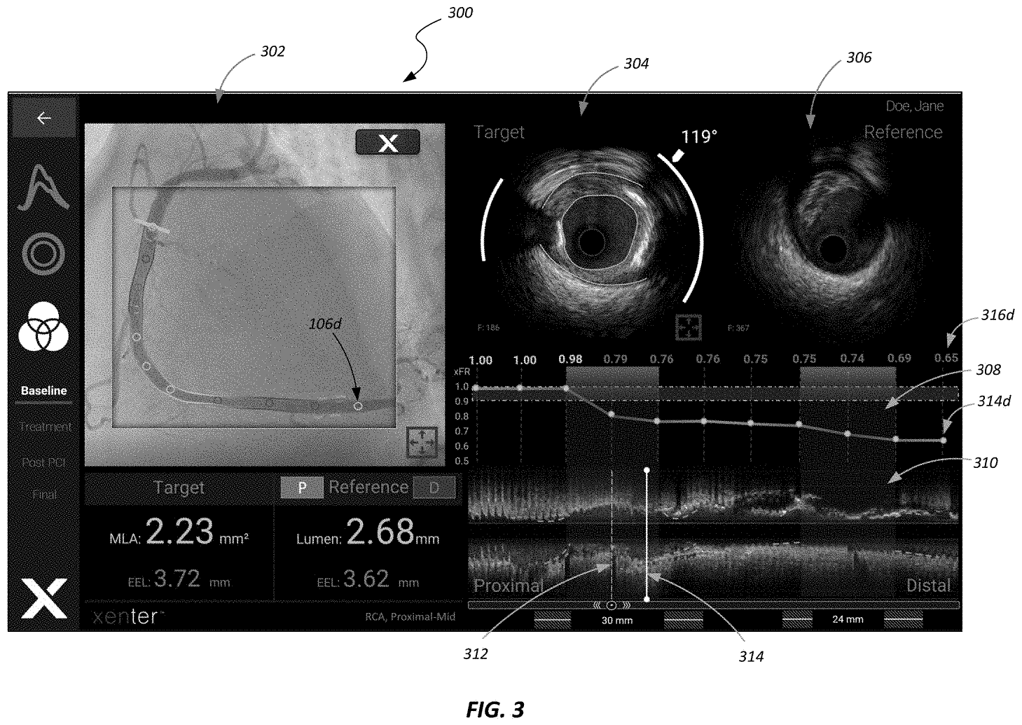

Having the pressure data from the guidewire system 100, the ultrasound imaging data from the catheter system 200, and knowing the relative positions of the catheter 202 and guidewire 102 when such data was obtained, all of the data may be correlated and presented to a practitioner (e.g., an interventional cardiologist) in a manner that enables them to make potential treatment decisions. For example, referring to FIG. 3, a screen shot is shown of a display 300 that correlates or “registers” multiple sets of data in a unique manner for the practitioner.

The display 300 may be provided by any suitable display system known in the art, including any suitable computer system comprising a monitor, television screen, projector, headset, or other display device. The display system may also include a computer device comprising one or more hardware storage devices and one or more processors. In some embodiments, the display system may comprise or be associated with the external device 110 and/or 220. The display system can be communicatively coupled to the guidewire system 100 and catheter system 200. For example, the display system can be communicatively coupled to control unit 112 and/or 212 (directly or through one or more communicatively coupled intermediate devices). The display system can also receive input through one or more input devices. Suitable input devices are known in the art and can include a mouse, keyboard, and/or touch screen, for example.

In the embodiment shown in FIG. 3, an angiogram image 302 is depicted of a patient's artery in the lefthand side of the display 300. In the upper righthand quadrant of the display, a pair of cross-sectional IVUS images of an artery are shown (i.e., the images are oriented to show a cross-sectional view that is perpendicular to the longitudinal axis of the artery) including a target image 304 and a reference image 306. In the lower righthand quadrant of the display 300, a graph representing a pressure map 308 of the artery is positioned above a longitudinal cross-sectional IVUS image 310 of the artery along a length of interest. The longitudinal IVUS image 310, while corresponding to a length of artery shown in the angiogram image 302, is shown extended along a substantially horizontal axis (i.e., the anatomical bends or curves shown in the angiogram image 310 have been “straightened” out).

The pressure map 308 is scaled and correlated with the longitudinal IVUS image 310 so that the representation of a physiological parameter (e.g., a pressure measurement, a pressure difference, or a pressure ratio) aligns with the location within the artery where a pressure measurement was taken. For example, in the embodiment depicted in FIG. 3, each dot in the pressure map 308 represents a sensor 106 on the guidewire 102. In one specific example, the dot 316d corresponds with the sensor 106d as also depicted in the angiogram image 302. Directly above each dot (e.g., 316d) is a physiological parameter (e.g., 318d which reads 0.65), in this case, a ratio of the pressure measurement taken by that particular sensor (e.g., distal-most sensor 106d) divided by the aortic pressure (e.g., the pressure taken by the proximal sensor 108). Viewing the longitudinal IVUS image 310 in direct alignment with a given dot of the pressure map 308 provides an ultrasound image indicating the condition of the artery at the longitudinal position of the associated sensor 106.

Looking at the pressure map 308, the dot furthest to the left represents the proximal-most sensor 106p of the distal group of sensors 106. The value above that dot is 1.00, indicating (in the example shown) that the pressure measured by the proximal most sensor 106p is the same as the pressure measured by the proximal sensor 108 or other aortic pressure sensor. As noted above, the dot furthest to the right in the pressure map 308 (i.e., dot 316d) represents the distal most sensor 106d of the distal group of sensors 106. Again, the value above that dot is 0.65 which indicates that the pressure measured by the distal-most sensor 106d is only about 65% of the pressure measured by the proximal sensor 108 (or other aortic pressure sensor). Other dots are representative of pressures measured by the remaining sensors 106, relative to the measured aortic pressure, at their respective locations within the artery.

Additionally, circles or dots (or other symbols or indicia) may be similarly overlayed onto the angiogram image 302 to show the locations where individual sensors took a pressure measurement within the artery on that image. For example, the lower-most right circle in the angiogram image 302 is representative of distal-most sensor 106d and its location within the artery when pressure measurements were taken. Other circles similarly correspond to the dots on the pressure map 308 (and thus, to associated sensors 106 on the guidewire 102).

Several additional aids are provided in association with the display 300 for assistance in assessing and treating an artery. For example, color coded annotation on the angiogram image 302 (applied to the image after a processing unit considers the various imaging data and pressure data) may indicate locations of lesions or stenosis that are recommended to be treated in some way. In FIG. 3, the segments depicted in the color red on the annotated angiogram image 302 (and the red graph lines on the pressure map 308) indicate that such areas are recommended to be treated (e.g., areas to be stented). On the aligned pressure map 308 and the longitudinal IVUS image 310, the purple or magenta shaded areas or bands correspond generally with the red lines on the angiogram image 302 and the pressure map 308. In FIG. 3, the segments depicted in the color blue on the annotated angiogram image 302 (and the corresponding blue graph lines on the pressure map 308) indicate areas that are considered to be satisfactory and that do not require intervention or treatment. In FIG. 3, the green dashed lines (both on the angiogram image 302 and the longitudinal IVUS image 310) indicate potential “landing areas” for the ends of stent. Considering the suggested landing areas, proposed stent lengths may also be displayed as stent length indicators, such as shown below the longitudinal IVUS image 310 as indicated by the “30 mm” bar and the “24 mm” bar. Of course, other color schemes or indicia may be used to convey such information, and the example above is not to be taken as limiting. Moreover, the particular layout of the different components of display 300 (e.g., the angiogram image 302, target image 304, reference image 306, pressure map 308, and the longitudinal IVUS image 310) may be varied, provided that the pressure map 308 and the longitudinal IVUS image 310 are preferably aligned as described above.

A variety of other information may be provided on the display 300 as well. For example, the MLA (minimum luminal area) of a vessel may be shown, indicating the cross-sectional area of a vessel within a lesion (shown as 2.23 mm² in this non-limiting example). The lumen diameter at a specific location (e.g., at the location being shown in the target IVUS image 304) may be displayed (shown as 2.68 mm in this non-limiting example). Other information may include external elastic lamina (EEL) measurements (shown as 3.72 mm and 3.62 mm in this non-limiting example), which is a benchmark for use in determining the size of a stent to help ensure that a stent is not oversized for a particular area of an artery.

Looking at the target IVUS image 304, other annotations may be provided including, for example, an indication of a calcific arc (e.g., the white line having a dimension of) 119°) showing calcified areas of an artery. Additionally, other annotations may depict the interior lumen and/or boundaries of or between different layers of the artery.

In assessing an artery, a user of the system may manipulate indicia on the display 300 (via one or more input devices such as disclosed herein) to provide different views or access different sets of information. For example, annotations may be turned on and off (e.g., the annotations in the angiogram image 302 may be toggled on and off so that a practitioner may view the artery with or without the pressure overlays or other information). Additionally, a section line 312 may be positioned on the longitudinal IVUS image 310 (or on the angiogram image 302 or on both) indicating the location from where the target IVUS image 304 is taken. Similarly, a second section line 314 may be positioned on the longitudinal IVUS image 310 (or on the angiogram image 302 or on both) indicating the location from where the reference IVUS image 306 is taken. Either of these lines may be manipulated (e.g., moved using a touch screen or other input device) to provide a different target or reference image. Thus, for example, the first section line may be moved to the right or to the left along the longitudinal IVUS image 310 causing the target IVUS image 304 to update correspondingly. Zooming, panning and other manipulative functions may also be utilized.

The image shown in FIG. 3 may be saved, e.g., as a “baseline” or “pre-treatment” image. After a treatment has been conducted (e.g., placement of a stent at a lesion location), new data may be obtained (e.g., new pressure readings by the guidewire 102 and new IVUS images obtained by the catheter 202), and the new data may be compiled for viewing on an updated view of the display 300. The updated view may include similar types of views and information as depicted in FIG. 3 and enable a practitioner to assess whether a particular treatment (e.g., a placed stent) has accomplished the desired effect.

One of skill in the art will appreciate that various functions and methods described above, including the interface functions provided by display 300, may be performed in whole or in part by computer instructions (e.g., software stored on one or more hardware storage devices) executed by one or more computer processors. The one or more computer processors may be comprised by or otherwise communicatively associated with control unit 112 and/or 212. In some embodiments, the one or more processors and/or the one or more computer executable instructions may at least in part be stored and/or executed remotely (e.g., in the cloud). As such, the disclosed functions and methods may be executed locally, remotely, or through a distributed computing system.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

When the terms “about,” “approximately,” “substantially,” “roughly,” or the like are used in conjunction with a stated amount, value, or condition, it may be taken to mean an amount, value or condition that deviates by less than 15%, less than 10%, less than 5%, less than 1%, less than 0.1%, or less than 0.01% of the stated amount, value, or condition.

While the disclosed embodiments may be susceptible to various modifications and alternative forms, specific embodiments have been shown by way of example in the drawings and have been described in detail herein. It is noted that features, elements, or components of one embodiment may be combined with features, elements, or components of other embodiments without limitation. However, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A medical device comprising:
an elongated member having a distal portion configured to be disposed in an artery;
a first group of sensors comprising at least two sensors associated with the distal portion of the elongated member, the first group of sensors configured to detect a physiological parameter at at least two different locations within the artery during a single heartbeat.

2. The medical device of claim 1, wherein the elongated member comprises a guidewire.

3. The medical device of claim 2, wherein the at least two sensors are spaced apart by approximately 0.5 cm to approximately 2.0 cm using a center-to-center spacing.

4. The medical device of claim 2, wherein the group of sensors includes five to fifteen sensors spaced approximately 0.5 cm to approximately 1.5 cm apart from each other using a center-to-center spacing.

5. The medical device of claim 3, wherein the group of sensors includes a plurality of pressure sensors.

6. The medical device of claim 5, further comprising a proximal pressure sensor associated with the guidewire and positioned proximally of the first group of sensors.

7. The medical device of claims 6, wherein the proximal pressure sensor is configured to detect a pressure within the artery during the same single heartbeat as the first group of sensors.

8. A system comprising:
a guidewire having a group of pressure sensors associated with a distal portion of the guidewire;
a catheter having at least one ultrasound transducer configured to provide an ultrasound image of an artery;
a display system configured to receive pressure sensor data from the guidewire and ultrasound image data from the catheter, the display system comprising instructions that are executable to cause the display system to generate a display including:
an angiogram image of the artery,
a pressure map of pressure measurements received from the group of pressure sensors, and
a longitudinal ultrasound image of the artery aligned with the pressure map.

9. The system of claim 8, wherein the display further comprises a first cross-sectional ultrasound image of the artery.

10. The system of claim 9, wherein the display further comprises a second cross-sectional ultrasound image of the artery.

11. The system of claim 10, wherein the first cross-sectional ultrasound image is of an area of the artery including a lesion.

12. The system of claim 11, wherein the second cross-sectional ultrasound image is a reference image for comparison with the first cross-sectional image.

13. The system of claim 8, wherein the display further comprises annotations overlaid on the angiogram image, the annotations including indications of suggested landing zones for a stent.

14. The system of claim 8, wherein the display further comprises indicia representing a suggested stent length positioned in alignment with an indication of a lesion on the longitudinal ultrasound image.

15. The system of claim 8, wherein the group of pressure sensors includes at least two sensors spaced apart between approximately 0.5 cm and approximately 2.0 cm using a center-to-center spacing.

16. The system of claim 8, wherein the group of pressure sensors includes five to fifteen sensors spaced approximately 0.5 cm to approximately 1.5 cm apart from each other using a center-to-center spacing.

17. The system of claim 8, wherein the group of pressure sensors is configured to detect a physiological parameter at at least two different locations within the artery during a single heartbeat of a patient.

18. The system of claim 17, further comprising a proximal pressure sensor.

19. The system of claim 18, wherein the proximal pressure sensor is configured to detect a pressure within the artery during the same single heartbeat as the first group of sensors.

20. The system of claim 19, wherein the proximal pressure sensor is associated with the guidewire and located proximally of the group of pressure sensors.
